(19) United States
(12) Patent Application Publication
(10) Pub. No.: US 2008/0003127 A1
(43) Pub. Date: Jan. 3, 2008

(54) Non-Ferrous Metal Cover Gases

(75) Inventors: Matthew H. Luly, Hamburg, NY (US); Rajiv R. Singh, Getzville, NY (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International Inc., Morristown, NJ (US)

(21) Appl. No.: 11/691,276

(22) Filed: Mar. 26, 2007

Related U.S. Application Data
(60) Provisional application No. 60/818,416, filed on Jul. 3, 2006.

(51) Int. Cl. C22B 26/12; C07C 21/18; C22B 26/22

(52) U.S. Cl. 420/400; 420/402; 420/528; 570/135; 570/136; 75/300; 75/602; 75/680; 75/745

(57) ABSTRACT

Disclosed are cover gas compositions comprising fluoroolefins for impeding the oxidation of molten nonferrous metals and alloys, such as magnesium.

### CROSS REFERENCE TO RELATED APPLICATION

This application claims the priority of U.S. Provisional Application 60/818,416, filed on Jul. 3, 2006. The contents of this provisional application are incorporated herein by reference.

### BACKGROUND OF THE INVENTION

(1) Field of Invention

The present invention relates to cover gas compositions for molten nonferrous metal, such as magnesium, and methods of using the same to prevent the oxidation when the metal is exposed to air.

(2) Description of Related Art

Certain non-ferrous metals, such as magnesium, aluminum, and lithium, are highly reactive and oxidatively unstable. For example, molten magnesium is readily and violently oxidized in ambient air or dry air, burning with a flame temperature of approximately 2820° C. Three approaches have been suggested to inhibit these severe oxidation processes: (1) sprinkling salt cover fluxes over the molten metal; (2) excluding oxygen from contacting the molten metal by blanketing the molten metal with an inert gas such as helium, nitrogen or argon; or (3) blanketing the molten metal with a protective cover gas composition. Protective cover gas compositions typically comprise air and/or carbon dioxide and a small amount of an inhibiting agent which reacts or interacts with the molten metal to form a film or layer on the molten metal surface which protects it from oxidation.

U.S. Pat. No. 1,972,317 (Reimers) relates to methods for inhibiting the oxidation of readily oxidizable metals, including magnesium and its alloys. Reimers notes that at the time of its filing in 1932, numerous solutions had been proposed to the oxidation problem including displacing the atmosphere in contact with the metal with a gas such as nitrogen, carbon dioxide, or sulfur dioxide. Reimers teaches inhibition of oxidation by maintaining in the atmosphere in contact with molten metal an inhibiting gas containing fluorine, either in elemental or combined form. Reference is made to many fluorine containing compounds with the solids ammonium borofluoride, ammonium silicofluoride, ammonium bifluoride and ammonium fluophosphate or the gases evolved therefrom upon heating being said to be preferred. Notwithstanding the disclosure in Reimers, it was not until about the mid-1970's that a fluorine containing compound found commercial acceptance as an inhibiting agent in a cover gas.

Prior to about the mid-1970's, sulfur dioxide (SO₂) was widely used as an inhibiting agent in a magnesium cover gas composition. However, SO₂ was subsequently replaced by sulfurhexafluoride (SF₆) which is currently the industry standard. Typically, SF₆ based cover gas compositions contain 0.2-1% by volume SF₆ and a carrier gas such as air, carbon dioxide, argon, or nitrogen. SF₆ has the advantages that it is a colorless, odorless, non-toxic gas which can be used for protecting molten magnesium/magnesium alloy and in the production of bright and shiny ingots with relatively low dross formation. However, SF₆ suffers from several disadvantages, including: its sulfur-based decomposition products at high temperature are very toxic; it is expensive and has limited sources of supply; and it is a known greenhouse gas having, at a time horizon of 100 years, a Global Warming Potential (GWP) of 23,900 relative to 1 for carbon dioxide.

It is also noted that once magnesium has ignited, the resulting fire cannot be extinguished even with high concentrations of SF₆. The potential byproduct SO, is even worse in this respect as it can accelerate a magnesium fire.

Another cover gas useful for extinguishing a magnesium fire is boron trifluoride (BF₃). However, this material tends to be very expensive and is also very toxic.

The problem of GWP of cover gases has been addressed in WO 00/64614 wherein certain relatively low GWP hydrofluorocarbons and hydrofluoroethers such as difluoromethane (HFC-32), pentafluoroethane (HFC-125), 1,1,1,2-tetrafluoroethane (HFC-134a), difluoroethane (HFC-152a), methoxy-nonafluorobutane (HFE-7100), ethoxy-nonafluorobutane (HFE-7200), and others were disclosed as being useful as blanket gases for protecting molten magnesium and magnesium alloys from oxidation. U.S. Pat. No. 6,521,018 (Hobbs) also discloses certain low GWP compounds that may be useful as blanket gases for nonferrous metals and alloys including, carbonyl fluoride (COF₂), trifluoroacetyl fluoride (CF₃COF), 1,1,1,3,3,3-hexafluoropropan-2-one ((CF₃)₂CO), nitrogen trifluoride (NF₃), sulfuryl fluoride (SO₂F₂), nitrosyl fluoride (NOF), fluorine gas (F₂), and others. Still other compounds useful for magnesium blanket gases are disclosed in U.S. Pat. No. 6,537,346, U.S. Pat. No. 6,685,764, and U.S. Pat. No. 6,780,220 (all by Milbrath), including perfluoroketones such as C₂F₅C(O)CF(CF₃)₂.

Although previously suggested compounds may have certain limited utility as cover gases, alternative cover gas compositions that have superior characteristics, such as a low GWP, low boiling point, uniform dispersement, and low or no toxicity, are desirable. Applicants have discovered that certain fluoroolefins, such as for example, CF₃CH═CHF (trans-HFO-1234ze), are useful as cover gases for nonferrous reactive metals. Applicants discovery is contrary, in at least some respects, to prior teachings. For example, it has heretofore been believed that fluoroolefins are undesirable as cover gases due to environmental and/or toxicity concerns. (See e.g. D. Milbrath, “Development of 3M Novec 612 Magnesium Protection Fluid as a Substitute for SF6 over Molten Magnesium”, International Conference on SF6 and the Environment, Nov. 21-22, 2002, www.epa.gov/highgwp/electricpower-sf6/pdf/milbrath.pdf.)

### SUMMARY OF THE INVENTION

One aspect of the present invention provides compositions for impeding the oxidation of molten nonferrous metals and alloys, such as magnesium, when such metals are exposed to oxidation conditions, such as being exposed to an oxygen-containing gas (for example air). In certain embodiments, such compositions preferably comprise at least one fluoroolefin, more preferably at least one C2-C6 fluoroolefin, more preferably one or more C3 to C5 fluoroolefins, even more preferably one or more compounds having Formula I as follows:

XCF_{z}R_{3-z} (I)

where X is a C1, C2, C3, C4, or C5 unsaturated, substituted or unsubstituted,

radical, each R is independently Cl, F, Br, I or H, and z is 1 to 3. Most preferable fluoroolefins include trans-1,3,3,3-tetrafluoropropene (trans-HFO-1234ze), cis-1,1,1,2,3-pentafluoropropene (cis-HFO-1225ye), 3-chloro-1,1,1-trifluoropropene (HFCO-1233xf), cis-1,1,1-trifluoro-3-chloro-propene (cis-HFCO-1233zd), and trans-1,1,1-trifluoro-3-chloro-propene (trans-HFCO-1233zd).

In certain preferred embodiments the fluoroalkene of the present invention has at least four (4) halogen substituents, at least three of which are F. In certain embodiments, the compound of the present invention does not include any Br substituents.

For embodiments in which at least one Br substituent is present, it is preferred that the compound includes no hydrogen. In such embodiments it also generally preferred that the Br substituent is on an unsaturated carbon, and even more preferably the Br substituent is on a non-terminal unsaturated carbon. One particularly preferred compound in this class is CF₃CBr═CF₂, including all of its isomers.

In certain embodiments it is highly preferred that the compounds of Formula I are propenes, butenes, pentenes and hexenes having from 3 to 5 fluorine substituents, with other substituents being either present or not present. In certain preferred embodiments, no R is Br, and preferably the unsaturated radical contains no Br substituents. Among the propenes, fluorochloroporpenes (such as trifluoro, monochloropropenes (HFCO-1233)), and even more preferably CF₃CCl═CH₂ (HFCO-1233xf), cis-CF₃CH═CHCl (HFCO-1233zd), and trans-CF₃CH═CHCl (HFCO-1233zd), and are especially preferred in certain embodiments.

In certain embodiments, pentafluoropropenes are preferred, including particularly those pentafluoropropenes in which there is a hydrogen substituent on the terminal unsaturated carbon, such as cis-CF₃CF═CFH(HFO-1225ye), particularly since applicants have discovered that such compounds have a relatively low degree of toxicity in comparison to at least the compound CF₃CH═CF₂ (HFO-1225zc).

Among the butenes, fluorochlorobutenes are especially preferred in certain embodiments.

The term “HFO-1234” is used herein to refer to all tetrafluoropropenes. Among the tetrafluoropropenes are included 1,1,1,2-tetrafluoropropene (HFO-1234yf) and both cis- and trans-1,1,1,3-tetrafluoropropene (HFO-1234ze). The term HFO-1234ze is used herein generically to refer to 1,1,1,3-tetrafluoropropene, independent of whether it is the cis- or trans-form. The terms “cis-HFO-1234ze” and “trans-HFO-1234ze” are used herein to describe the cis- and trans-forms of 1,1,1,3-tetrafluoropropene respectively. The term “HFO-1234ze” therefore includes within its scope cis-HFO-1234ze, trans-HFO-1234ze, and all combinations and mixtures of these.

The term “HFCO-1233” is used herein to refer to all trifluoro-monochloropropenes. Among the trifluoro-monochloropropenes are included 1,1,1-trifluoro-2-chloro-propene (HFCO-1233xf) and both cis- and trans-1,1,1-trifluo-3-chlororopropene (HFCO-1233zd). The term HFCO-1233zd is used herein generically to refer to 1,1,1-trifluo-3-chloropropene, independent of whether it is the cis- or trans-form. The terms “cis-HFCO-1233zd” and “trans-HFCO-1233zd” are used herein to describe the cis- and trans-forms of 1,1,1-trifluo-3-chlororopropene, respectively. The term “HFCO-1233zd” therefore includes within its scope cis-HFCO-1233zd, trans-HFCO-1233zd, and all combinations and mixtures of these.

The term “HFO-1225” is used herein to refer to all pentafluoropropenes. Among such molecules are included 1,1,1,2,3 pentafluoropropene (HFO-1225ye), both cis- and trans-forms thereof. The term HFO-1225ye is thus used herein generically to refer to 1,1,1,2,3 pentafluoropropene, independent of whether it is the cis- or trans-form. The term “HFO-1225ye” therefore includes within its scope cis-HFO-1225ye, trans-HFO-1225ye, and all combinations and mixtures of these.

The present invention provides also methods and systems which utilize the compositions of the present invention, including methods and systems for preventing oxidation of molten nonferrous metals.

As used herein, the term “air” means either ambient air, dry air, or moist air. Such compounds advantageously have an exceptionally low GWP potential, a relatively low boiling point, and are relatively non-toxic.

In addition, this invention relates to molten reactive metal having a protective film on its surface that is formed by a reaction between the metal and a composition containing an effective amount of fluoroolefin of the present invention, preferably said amount being effective under the intended circumstances to at least partially passivate the surface of the metal, thereby reducing the chemical reactivity of the metal, especially the metal's oxidative reactivity.

According to another aspect of the present invention, provided is a method for impeding the oxidation of a molten nonferrous metal exposed to and oxygen-containing gas, such as air, comprising: (a) providing molten nonferrous metal, such as magnesium, having a surface; (b) exposing said surface to a fluoroolefin composition of the present invention, preferably a gaseous form of such a composition, and even more preferably a gas containing one or more of trans-HFO-1234ze, cis-HFO-1225ye, HFCO-1233xf, cis-HFCO-1233zd, and trans-HFCO-1233zd; and optionally (c) forming an oxidized film on said surface. In certain preferred aspects of the method, the exposed surface of the molten reactive metal is exposed to or contacted with the gaseous fluoroolefin composition. Without being bound by or to any particular theory of operation, it is believed that the fluoroolefin composition in preferred embodiments reacts with the metal to produce an oxidatively stable film on its surface. By forming this film, the oxygen in the air can be effectively separated from the surface of the molten reactive metal and thus prevent or at least substantially inhibit the oxidation of the metal by the oxygen.

According to yet another aspect of the present invention, provided is a method for extinguishing a fire on a surface of a molten nonferrous metal, such as magnesium, comprising contacting said surface with a gaseous fluoroolefin composition of the present invention, including preferably a gaseous composition comprising one or more tetrafluoropropene, such as trans-HFO-1234ze, cis-HFO-1225ye, HFC-1233xf, cis-HFCO-1233zd, and trans-HFCO-1233zd.

### DETAILED DESCRIPTION OF PREFERRED EMBODIMENTS OF THE INVENTION

The fluoroolefin compositions of the present invention are generally effective as cover gases to impede the oxidation of molten reactive metals when the surface of the metal is exposed to source of oxygen, such as air. As used herein, the term “nonferrous reactive metal” means a metal or alloy which is sensitive to destructive, vigorous oxidation when exposed to air, such as magnesium, aluminum, or lithium, or an alloy comprising at least one of these metals. For convenience, the following description of illustrative embodiments of the invention shall refer to magnesium. It is understood, however, that the present invention can also be used with aluminum, lithium, or other nonferrous reactive metal, or an alloy containing at least one of these metals.

Without necessarily being bound by theory, it is believed that by impeding oxidation, the cover gas composition of the present invention is capable of protecting the molten metal from ignition. As is the case with known fluorine-containing cover gases, it is believed that the fluoroolefin compositions of the present invention can react with the molten metal surface to create a thin passivation layer or film that can function as a barrier between the metal and an oxygen source. In contrast to conventional fluorine compounds that are used in cover gases, the fluoroolefins of the present invention are particular advantageous in that they have a relatively low GWP and a relatively low atmospheric lifetime, while also being non-toxic, effective at low concentrations, and have a low boiling point.

In certain preferred embodiments, the compositions of the present invention comprise fluoroolefins consisting of carbon, fluorine, and optionally hydrogen atoms. In certain preferred embodiments, the fluoroolefins are selected from a C₂-C₄ perflorinated olefin. However, more preferred are C₂-C₄ fluoroolefins having at least one hydrogen atom. Examples of preferred fluoroolefins include, but are not limited to, trans-HFO-1234ze, cis-HFO-1225ye, HFC-1233xf, cis-HFCO-1233zd, and trans-HFCO-1233zd.

Fluoroolefin compositions of the present invention may include a mixture of at least one fluoroolefin and, optionally, a carrier gas. Preferred carrier gases include, but are not limited to, nitrogen, carbon dioxide, air, and/or noble gas such as argon. Preferably, the composition comprises a minor amount of at least one fluoroolefin and a major amount of a carrier gas. In certain preferred embodiments, the composition comprises from about 0.01 to about 2 weight percent of at least one fluoroolefin and from about 99.99 to about 98 weight percent of a carrier gas.

As used herein, “GWP” is a relative measure of the warming potential of a compound based on the structure of the compound. The concept of GWP was developed to compare the ability of each greenhouse gas to trap heat in the atmosphere relative to another gas. Generally, the GWP for a particular greenhouse gas is the ratio of heat trapped by one unit mass of the greenhouse gas to that of one unit mass of CO₂ over a specified time period. More specifically, the GWP of a compound, as defined by the Intergovernmental Panel on Climate Change (IPCC) in 1990 and updated in Scientific Assessment of Ozone Depletion: 1998 (World Meteorological Organization, Scientific Assessment of Ozone Depletion: 1998, Global Ozone Research and Monitoring Project—Report No. 44, Geneva, 1999), is calculated as the warming due to the release of 1 kilogram of a compound relative to the warming due to the release of 1 kilogram of CO₂ over a specified integration time horizon (ITH):

$${GWP}_{X} ( {t}^{′} ) = \frac{{\int }_{0}^{{t}^{′}} {F}_{X} exp ( - t / {τ}_{X} ) d t}{{\int }_{0}^{{t}^{′}} {F}_{{CO}_{2}} R ( t ) d t}$$

where F is the radiative forcing per unit mass of a compound (the change in the flux of radiation through the atmosphere due to the IR absorbance of that compound), C is the atmospheric concentration of a compound, τ is the atmospheric lifetime of a compound, t is time, and x is the compound of interest.

The commonly accepted ITH is 100 years representing a compromise between short-term effects (20 years) and longer-term effects (500 years or longer). The concentration of an organic compound, x, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

The cover gas compositions of the present invention preferably include those compositions wherein the fluoroolefin compounds included therein have a GWP of less than about 1000, more preferably less that about 150 and even more preferably of less than about 100. In certain preferred embodiments, each component present in the composition in a substantial amount has a GWP of less than about 1000, more preferably less that about 150 and even more preferably of less than about 100. In certain highly preferred embodiments, each component of the composition which is present in more than an insubstantial amount has a GWP of less than about 10, and even more preferably less than about 5. For comparison, the GWP of CO₂, certain conventional cover gases, and certain cover gases according to the present invention are shown in Table A.

TABLE A

| | GWP | Atmospheric | Boiling Point |
|---|---|---|---|
| Compound | (100 Yr) | Lifetime (Yr) | (° C.) |
| CO₂ | 1 | 100-150 | −78 |
| SF₆ | 23,900 | 3,200 | −82 |
| NF₃ | 10,800 | 740 | −121 |
| C₂F₆ | 11,400 | 10,000 | −78 |
| HFC-134a | 1600 | 13.6 | −26 |
| HFC-152a | 140 | 1.5 | −25 |
| HFE-7100 | 320 | 4.1 | 61 |
| SO₂F₂ | 0.1 | dissipates quickly | −55 |
| | | via hydrolysis | |
| | | and photodegradation | |
| C₂F₅C(O)CF(CF₃)₂ | 10 | 0.1 | 49 |
| HFO-1234yf | 4 | 0.04 | −30 |
| trans-HFO-1234ze | 6 | 0.05 | −18.4 |
| cis-HFO-1234ye | <15 | <0.1 | +2 |
| HFCO-1233xf | <20 | <0.1 | +12 |
| cis-HFCO-1233zd | <20 | <0.1 | +19 |
| trans-HFCO-1233zd | <20 | <0.1 | +19 |

Preferably, the cover gas compositions of the present invention include those compositions wherein each fluoroolefin component has a atmospheric lifetime of less than about 20 (years), preferably less than about 10 (years), and even more preferably less than about 1 (year). As used herein, the term “atmospheric lifetime” is the approximate amount of time it would take for the concentration of the compound to fall to e⁻¹ of its initial value as a result of either being converted into another chemical compound (wherein e is the base of natural logarithms). Atmospheric lifetime is closely related to GWP since relatively short lifetimes limit the duration that a reactant can participate in a reaction.

In addition, preferred cover gas compositions of the present invention comprise what are more compounds wherein each compound present in more than an insubstantial amount has a boiling point of less than about 25° C., and even more preferably less than about 0° C. Cover gases that have boiling points close to or above room temperature (i.e. which are liquids at room temperature) typically require additional metering equipment to disperse the cover gas material in a controlled fashion onto the surface of the molten metal.

Preferably, fluoroolefins used in the present compositions have low or no toxicity. In this regard, it is preferred that fluoroolefin components that a present in the compositions in more than an insubstantial amount have a LC-50 value of at least about 100,000 ppm, and more preferably at least about 200,000 ppm. As used herein, the term “LC-50 value” means the concentration of the fluoroolefin in air that will kill 50% of test subject (e.g. mice) when administered as a single exposure (e.g. 4 hours). For example, HFC-1234ze has been found to have a 4-hour LC-50 of at least 100,000, and HFC-1234yf has been found to have a 4-hour LC-50 of at least about 200,000. For comparison, C₂F₅C(O)CF(CF₃)₂ (a fluoroketone cover gas marketed by Minnesota Mining and Manufacturing Co. of St. Paul, Minn., under the tradename Novec™) has a 4-hour LC-50 of about 100,000. Other compounds, such as sulfuryl fluoride, nitrosyl fluoride, and nitrogen trifluoride are known to be toxic and/or hazardous materials.

Another measure of toxicity is a compound's No Observed Adverse Effect Level (NOAEL). As used herein, the term NOAEL refers to the greatest concentration or amount of a substance, found by experiment or observation, which causes no detectable adverse alteration of morphology, functional capacity, growth, development, or life span of the target organism under defined conditions of exposure. For cardiac sensitization tests, the NOAEL for HFO-1234yf and HFO-1234ze are greater than 12 vol. %. By comparison, the NOAEL for C₂F₅C(O)CF(CF₃)₂ is only 10 vol. %.

Applicants have found that different isomeric forms of certain fluoroolefins do not possess the same advantageous characteristics for cover gas applications. For example, among isomers of HFO-1225, the HFO-1225zc isomer is much more toxic, and thus less preferred, than the HFO-1225ye or HFO-1225yc isomer. In certain preferred embodiments, the cover gas consists essentially of only a single isomer of fluoroolefin. For example, in certain embodiments the trans-isomer of HFO-1234ze can be utilized in the present invention with much greater success than the related cis-isomer or than mixtures of the cis- and trans-isomers. In particular, the trans-isomer is more preferred not only because is less toxic than the cis-isomer, but also because it has a lower normal boiling point (−18.4° C. vs. 9° C. for trans- and cis-isomers, respectively). This low boiling point correlates to a higher vapor pressure of the gas which is advantageous in that the gas is more easily metered as it is applied to a molten metal. Isomeric mixtures of the cis- and trans-isomers can be problematic because the isomers do not have the same vapor pressure, and thus are not evenly dispensed from a container. That is, dispersement of the isomeric mixture from a container will initially result in a cover gas having a higher concentration of the lower boiling isomer and will eventually result in a cover gas having a higher concentration of the higher boiling isomer. Such a mixture makes it more difficult to maintain a steady flow and composition.

### EXAMPLES

Certain aspects of the present invention are further illustrated, but is not limited by, the following examples.

Examples 1-5 demonstrate the efficacy of a fluoroolefin as a Mg cover gas according to the present invention.

### Example 1

A quartz tube having a well was equipped with a metered source of cover gas and a thermocouple which was placed in the well. The well was filled with about 0.2 to 0.3 g of solid magnesium pieces. The cover gas was a mixture of air (a carrier gas) and trans-HFO-1234ze. The air and the trans-HFO-1234ze were provided from separate cylinders and the relative amounts of each entering the mixture were controlled to give composition of about 4.5% trans-HFO-1234ze by volume.

The tube containing the magnesium was placed in an oven. A flow of cover gas through the tube and over the well containing the magnesium was then established at about 1 liter/minute. The oven was then heated to about 700° C. The flow of cover gas proceeded until a surface film was formed on the magnesium or the magnesium ignited.

After the test was complete, the magnesium was removed from the oven and visually inspected to determine the quality of the cover gas.

The magnesium contained a white coating (presumably MgO or MgF₂) indicating that the magnesium was well protected.

### Example 2

The experiment of Example 1 was repeated, except that the cover gas contained about 1.5% trans-HFO-1234ze by volume.

The magnesium contained a white coating and the pieces were not stuck together indicating that the magnesium was well protected.

### Example 3

The experiment of Example 1 was repeated, except that the cover gas contained about 0.5% trans-HFO-1234ze by volume.

The magnesium contained a white coating and the pieces were not stuck together indicating that the magnesium was well protected.

### Example 4

The experiment of Example 1 was repeated, except that the cover gas contained about 0.2% trans-HFO-1234ze by volume.

The magnesium contained a white coating with some dark spots and the pieces were not stuck together indicating that the magnesium was well protected.

### Example 5

The experiment of Example 1 was repeated, except that the cover gas contained about 0.1% trans-HFO-1234ze by volume.

The magnesium contained a white coating with a few brown specks indicating that the magnesium was protected in general.

### Comparative Examples

The experiments of Examples 1-5 were repeated, except that the cover gas contained about either SF₆ or HFC-134a.

The results of the comparative examples are provided in Table B. In general, trans-HFO-1234ze, SF₆, and HFC-134a performed well as cover gases at concentrations at or above about 1.5% by volume. However, performance of the different cover gases began to vary at about 0.5% by volume, with HFC-134a performing better than SF₆, and trans-HFO-1234ze performing better than HFC-134a. It is believed that the ability of the cover gas to protect the magnesium, and particularly to keep the magnesium from igniting, corresponds to the amount of fluorine it provides to create a protective barrier. Thus, cover gases that are more reactive, such as trans-HFO-1234ze, are better suited to protect magnesium compared to more stable gases, such as SF₆.

TABLE B

| Vol. % of | | |
|---|---|---|
| F-Source | | |
| in Air | F-Source | Quality of Mg Protection |
| 4.5 | SF₆ | white coating; pieces not stuck together |
| 4.5 | HFC-134a | white coating; pieces not stuck together |
| 4.5 | trans-HFO- | white coating; pieces not stuck together |
| | 1234ze | |
| 1.5 | SF₆ | white coating; pieces not stuck together |
| 1.5 | HFC-134a | white coating; pieces not stuck together |
| 1.5 | trans-HFO- | white coating; pieces not stuck together |
| | 1234ze | |
| 0.43 | SF₆ | coating less white; brownish regions; |
| | | Mg maintained partial luster |
| 0.60 | HFC-134a | white coating with no brown spots |
| 0.46 | trans-HFO- | white coating with no brown spots |
| | 1234ze | |
| 0.20 | SF₆ | several brownish regions, very little luster, |
| | | Mg pieces stuck together |
| 0.18 | HFC-134a | white with brown spots, a couple of |
| | | pieces stuck together |
| 0.26 | trans-HFO- | white with dark spots, no pieces stuck together |
| | 1234ze | |
| 0.11 | SF₆ | failure; Mg ignited |
| 0.10 | HFC-134a | most brown specks, protected in general |
| 0.09 | trans-HFO- | a few brown specks, well protected in general |
| | 1234ze | |

Having thus described a few particular embodiments of the invention, various alterations, modifications, and improvements will readily occur to those skilled in the art. Such alterations, modifications, and improvements, as are made obvious by this disclosure, are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A cover gas composition for impeding the oxidation of molten nonferrous metals and alloys when exposed to air, said composition comprising at least one fluoroolefin.

2. The cover gas composition of claim 1 wherein said fluoroolefin is a C₂-C₆ fluoroolefin.

3. The composition of claim 2 wherein said C₂-C₆ fluorolefin comprises one or more C₃-C₅ fluoroolefins.

4. The composition of claim 2 wherein said C₂-C₆ fluorolefin comprises one or more compounds having Formula I:
XCF_{z}R_{3-z} (I)
where X is a C₁, C₂, C₃, C₄, or C₅ unsaturated, substituted or unsubstituted, radical, each R is independently Cl, F, Br, I or H, and z is 1 to 3.

5. The composition of claim 4 wherein said at least on of said compounds of Formula I has at least four (4) halogen substituents.

6. The composition of claim 4 wherein said one or more compounds of Formula I has at least three fluorine substituents.

7. The composition of claim 4 wherein said composition contains at least one compound of Formula I having no Br substituents.

8. The composition of claim 4 wherein said composition contains at least one compound of Formula I having at least one Br substituents and wherein said compound includes no hydrogen.

9. The composition of claim 4 wherein said composition contains at least one compound of Formula I having at least one Br substituent on an unsaturated carbon.

10. The composition of claim 4 wherein said composition contains at least one compound of Formula I having at least one Br substituent on a non-terminal unsaturated carbon.

11. The composition of claim 4 wherein said at least one compound of Formula I comprises at least one tetrafluoropropene.

12. The composition of claim 4 wherein said at least one compound of Formula I comprises at least one fluorochloroporpene.

13. The composition of claim 4 wherein said at least one compound of Formula I comprises at least one pentafluoropropene.

14. The composition of claim 4 wherein said pentafluoropropene has at least one hydrogen substituent on a terminal unsaturated carbon.

15. The cover gas composition of claim 4 wherein said fluoroolefin is selected from the group consisting of CF₃CF═CH₂, CF₃CH═CHF, and CF₃CF═CHF.

16. The cover gas composition of claim 1 wherein said composition further comprises at least one carrier gas selected from the group consisting of nitrogen, carbon dioxide, air, noble gas, and mixtures thereof.

17. The cover gas composition of claim 16 comprising from about 98 to about 99.99 weight percent of a carrier gas and from about 0.01 to about 2 weight percent of one or more fluoroolefins.

18. The cover gas composition of claim 1 wherein said metal is selected from the group consisting of magnesium, aluminum, lithium, and alloys thereof.

19. The cover gas composition of claim 18 wherein said metal is magnesium.

20. A method for impeding the oxidation of a molten nonferrous metal exposed to air, comprising:
(a) providing molten nonferrous metal having a surface; and
(b) exposing said surface to a layer of gaseous fluoroolefins composition.

21. The method of claim 20 further comprising the step of:
(c) forming an oxidized film on said surface.

22. The method of claim 20 wherein said fluoroolefin is a C₂-C₆ fluorolefin comprises one or more compounds having Formula I:
XCF_{z}R_{3-z} (I)
where X is a C₁, C₂, C₃, C₄, or C₅ unsaturated, substituted or unsubstituted, radical, each R is independently Cl, F, Br, I or H, and z is 1 to 3.

23. The method of claim 21 wherein said fluoroolefin is selected from the group consisting of CF₃CF═CH₂, CF₃CH═CHF, and CF₃CF═CHF.

24. The method of claim 20 wherein said fluoroolefin composition further comprises at least one carrier gas selected from the group consisting of nitrogen, carbon dioxide, air, noble gas, and mixtures thereof.

25. The method of claim 20 wherein said metal is selected from the group consisting of magnesium, aluminum, lithium, and alloys thereof.

26. The method of claim 25 wherein said metal is magnesium.

27. A molten metal composition comprising a nonferrous reactive metal having a protective film on its surface, wherein said film is formed by a reaction between the metal and a fluoroolefin composition and said film impedes the oxidation of said metal.

28. The molten metal composition of claim 27 wherein said metal is selected from the group consisting of magnesium, aluminum, lithium, and alloys of at least one these.

29. The molten metal composition of claim 28 wherein said metal is magnesium or a magnesium alloy.

30. The molten metal composition of claim 21 wherein said fluoroolefin composition comprises a C₂-C₆ fluorolefin comprises one or more compounds having Formula I:
XCF_{z}R_{3-z} (I)
where X is a C₁, C₂, C₃, C₄, or C₅ unsaturated, substituted or unsubstituted, radical, each R is independently Cl, F, Br, I or H, and z is 1 to 3.

31. The molten metal composition of claim 30 wherein said C₂-C₆ fluoroolefin is selected from the group consisting of CF₃CF═CH₂, CF₃CH═CHF, and CF₃CF═CHF.

32. A cover gas composition for impeding the oxidation of molten nonferrous metals and alloys when exposed to air, said composition comprising at least one fluoroolefin selected from the group consisting of trans-1,3,3,3-tetrafluoropropene, cis-1,1,1,2,3-pentafluoropropene, and fluorochloroporpenes.

33. The cover gas of claim 32 wherein said fluoroolefin comprises trans-1,3,3,3-tetrafluoropropene.

34. The cover gas of claim 32 wherein said fluoroolefin consists essentially of trans-1,3,3,3-tetrafluoropropene.

35. The cover gas of claim 32 wherein said fluoroolefin consists of trans-1,3,3,3-tetrafluoropropene.

36. The cover gas of claim 32 wherein said fluoroolefin comprises cis-1,1,1,2,3-pentafluoropropene.

37. The cover gas of claim 32 wherein said fluoroolefin consists essentially of cis-1,1,1,2,3-pentafluoropropene.

38. The cover gas of claim 32 wherein said fluoroolefin consists of cis-1,1,1,2,3-pentafluoropropene.

39. The cover gas of claim 32 wherein said fluorochloroporpenes are trifluoro-monochloro-propenes.

40. The cover gas of claim 39 wherein said trifluoro-monochloro-propenes are selected from the group consisting of CF₃CCl═CH₂ (HFCO-1233xf), cis-CF₃CH═CHCl (HFCO-1233zd), and trans-CF₃CH═CHCl (HFCO-1233zd).

41. The cover gas composition of claim 32 wherein said composition further comprises at least one carrier gas selected from the group consisting of nitrogen, carbon dioxide, air, noble gas, and mixtures thereof.
